(19) United States
(12) Patent Application Publication
(10) Pub. No.: US 2017/0131298 A1
(43) Pub. Date: May 11, 2017

(54) Apparatuses, Methods and Compositions for Compound Detection Using Interfacial Nano-Biosensing in Microfluidic Droplets

(71) Applicant: XiuJun Li, El Paso, TX (US)

(72) Inventor: XiuJun Li, El Paso, TX (US)

(73) Assignee: THE BOARD OF REGENTS OF THE UNIVERSITY OF TEXAS SYSTEM, Austin, TX (US)

(21) Appl. No.: 15/345,005

(22) Filed: Nov. 7, 2016

Related U.S. Application Data
(60) Provisional application No. 62/252,197, filed on Nov. 6, 2015.

(51) Int. Cl. G01N 33/74; B01L 3/00

(52) U.S. Cl. CPC G01N 33/743; B01L 3/502715; B01L 3/502784; B01L 2300/0816; B01L 2300/0867; B01L 2300/0883; B01L 2200/0647; B01L 2300/0636

(57) ABSTRACT

A simple interfacial nano-biosensing strategy for high-sensitivity detection of low-solubility compounds like 17β-estradiol is disclosed. Apparatuses, methods and compositions for detection incorporate a combination of droplet microfluidics with aptamer-functionalized GO nanosensors.

### PRIORITY CLAIM

The present application claims priority to U.S. Application 62/252,197 filed Nov. 6, 2015 and is incorporated herein by reference.

### REFERENCE TO SEQUENCE LISTING

A sequence listing required by 37 CFR 1.821-1.825 is being submitted electronically with this application. The sequence listing is incorporated herein by reference.

### BACKGROUND

The present disclosure relates generally to apparatuses, methods, and compositions for compound detection using microfluidic lab-on-a-chip. More particularly, the disclosure relates to apparatuses, methods and compositions for detecting compounds, such as steroids, which have low water solubility, using a chip that includes interfacial nano-biosensing in microfluidic droplets.

The U.S. Environmental Protection Agency has noted that contaminants of emerging concern (CECs) in the environment may originate from many different sources. The chemical classes that comprise CECs include but are not limited to: flame-retardants, fluorinated alkyl phenol surfactants, phthalates bis-phenol A, steroids, hormones, pharmaceuticals, and various personal care products.

17β-estradiol, a sex hormone, is the most potent and ubiquitous member of the mammalian estrogenic steroids. However, 17β-estradiol is also an endocrine disrupting compound (EDC), and becomes one of the most potential environmental endogenous estrogens, due to its detrimental effects on endocrine function of human and aquatic organisms. Estradiol has a critical impact on reproductive and sexual functions, and affects the functions of other organs as well. Estradiol has been widely used in animal fattening because of its anabolic effects. But it is harmful to aquatic organisms and drastic problems can be caused through the food chain to human beings. Therefore, the rapid and sensitive detection of estradiol is of significance for environmental and food safety monitoring.

Furthermore, with the “doping” scandals present in various athletic events, drug screening using urinalysis has become routine. Low cost, rapid, and sensitive methods for detection of steroids in urine samples have broad applicability in any environment where there is concern about human use of such compounds.

Microfluidics is a relatively new technique in the diagnostic research field that offers a unique opportunity for various biomedical applications (Li and Li, 2010, Expert Review of Clinical Pharmacology, 3:267-80). Microfluidics provides for minimal reagent consumption, integrated processing, and analysis of complex fluids with high efficiency and sensitivity. As such, microfluidics is used to evaluate the presence of minute quantities of compounds that may be present in aqueous solutions such as biological fluids, water and food.

A broad range of molecules such as 17β-estradiol has limited solubility in aqueous solutions, which often affect their detection in many widely-used detection systems. Current methods for estradiol detection include high-performance liquid chromatography (HPLC), gas chromatography-mass spectrometry (GC-MS), and liquid chromatography-mass spectrometry (LC-MS). Despite the high sensitivity, these methods rely on sophisticated and expensive instruments, complicated sample preparation procedures, long assay time and are labor intensive. Recently, aptamer-based electrochemical biosensors and aptamer-based optical biosensor have been developed for simple estradiol detection. However, because 17β-estradiol is almost insoluble in water, water miscible organic solvents are required in these methods to dissolve estradiol, and the distribution ratio of each component needs to be carefully optimized to ensure that estradiol is completely dissolved. Thus, these assays usually require multi-step complicated procedures. These limitations compromise the advantages of detection simplicity from aptamers, and hinder the extensive application of such detection approaches. More importantly, because of insolubility of estradiol, the detection sensitivity of most methods is not high. Most limits of detection (LOD) for estradiol are in the range of nanomolar to subnanomolar (e.g. 2.1 nM), making them incapable of detecting trace contaminants of estradiol.

What is needed then, are simple, rapid, low cost, and sensitive microfluidics-based detection apparatuses, methods and compositions for compounds, such as 17β-estradiol, which are either insoluble or have low solubility in water.

### SUMMARY

In view of the aforementioned problems and trends, embodiments of the present invention provide systems, methods, and apparatuses for direct detection of compounds that have poor solubility in water, such as steroids, including 17β-estradiol. The compound to be detected can be soluble in a non-aqueous solvent. In certain aspects all or part of a sample containing or suspected of containing a target component is solubilized or introduced into a non-aqueous solvent stream. A probe complex is dissolved in an aqueous solution, which can be introduced into the non-aqueous solvent stream forming water-in-oil droplets that contain the probe complex. The probe complex is contacted with a component of the sample at the water/oil interface. The probe complex can bind the target component and produce a detectable signal. In certain aspects the probe complex is an aptamer/graphene oxide complex. In a further aspect the aptamer specifically binds a steroid molecule. In still a further aspects the steroid molecule is an estrogen, e.g., 17β estradiol. The probe complex can be connected to detectable molecule. In certain aspects the detectable molecule is a fluorescent molecule. In another aspect the fluorescent molecule is quench or non-fluorescent when complexed with GO.

According to a first aspect of the disclosure, a method for detecting water immiscible compounds, includes one or more of the steps of fabricating at least one microfluidic chip; generating microfluidic droplets by the addition of a sample, ethyl acetate, and an aptamer nanosensor complex; allowing competitive binding between the aptamer nanosensor complex and the sample; and measuring the level of fluorescence released following the competitive binding.

In a second aspect of the disclosure, the microfluidic chip includes two layers, wherein the first layer is a polymer, e.g., polydimethylsiloxane (PDMS), and the second layer is glass.

The first layer may further include at least one inlet for the delivery of the sample, at least one inlet for delivery of the ethyl acetate, and at least one inlet for delivery of the aptamer nanosensor complex, specifically, an aptamer-graphene oxide (GO) complex, a plurality of microchannels, and an outlet for droplet collection.

In another aspect of the disclosure, an apparatus for detecting water immiscible compounds includes one or more of means for fabricating at least one microfluidic chip; means for generating microfluidic droplets by the addition of a sample, ethyl acetate and an aptamer nanosensor complex; means for allowing competitive binding between the aptamer nanosensor complex and the sample; means for measuring the level of fluorescence released following the competitive binding.

In yet another aspect of the disclosure, compositions for detecting water immiscible compounds may include an aptamer-GO complex bound to estradiol.

Other aspects of the embodiments described herein will become apparent from the following description and the accompanying drawings, illustrating the principles of the embodiments by way of example only.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

Certain terms are used throughout the following description and claims to refer to particular system components and configurations. As one skilled in the art will appreciate, the same component may be referred to by different names. This document does not intend to distinguish between components that differ in name but not function.

The phrase “specifically binds” or “specifically immunoreactive” to a target refers to a binding reaction that is determinative of the presence of the molecule, microbe, or other targets in the presence of a heterogeneous population of other biologics. Thus, under designated conditions, a specified molecule binds preferentially to a particular target and does not bind in a significant amount to other biologics or components present in the sample.

As used herein, the term “sample” or “test sample” generally refers to a material suspected of containing one or more targets. The test sample may be used directly as obtained from the source or following a pretreatment to modify the character of the sample. The test sample may be derived from any environmental or biological source, such as an environmental or biological solid, fluid, or gas. The test sample may be pretreated prior to use, such as preparing plasma from blood, diluting viscous fluids, and the like. Methods of treatment may involve filtration, precipitation, dilution, distillation, mixing, concentration, inactivation of interfering components, and the addition of reagents. Besides biological fluids, other liquid samples may be used such as food products and the like for the performance of environmental or food production assays. In addition, a solid material suspected of containing the target may be used as the test sample. In some instances it may be beneficial to modify a solid test sample to form a liquid medium or to release a target.

Various embodiments of the devices described herein incorporate microchannels (microfluidic channels). The terms “microfluidic channel” or “microchannel” are used interchangeably and refer to a channel having at least one characteristic dimension (e.g., width or diameter) less than 1,000 more preferably less than about 900 μm, or less than about 800 μm, or less than about 700 μm, or less than about 600 μm, or less than about 500 μm, or less than about 400 μm, or less than about 300 μm, or less than about 250 μm, or less than about 200 μm, or less than about 150 μm, or less than about 100 μm, or less than about 75 μm, or less than about 50 μm, or less than about 40 μm, or less than about 30 μm, or less than about 20 μm.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates a composite schematic of a droplet microfluidic system. FIG. 1 shows a magnified view of portion (a) and portion (b) of the droplet microfluidic system.

FIG. 2A depicts a schematic of the droplet generation process while FIG. 2B is a fluorescence image after droplet generation using Cy3-labelled aptamers.

FIG. 3 is a graph depicting estradiol aptamer concentration optimization.

FIG. 4A depicts a composite of fluorescence images depicting the detection of different estradiol concentrations using a droplet microfluidic nanosensing system, while FIG. 4B is the calibration curve of this nanosensing system.

FIG. 5 is a graph depicting the comparison of estradiol detection results using the droplet microfluidic on-chip nanosensing system and two off-chip detection methods (a tube with shaking and a tube without shaking).

### DESCRIPTION

The foregoing description of the figures is provided for the convenience of the reader. It should be understood, however, that the embodiments are not limited to the precise arrangements and configurations shown in the figures. Also, the figures are not necessarily drawn to scale, and certain features may be shown exaggerated in scale or in generalized or schematic form, in the interest of clarity and conciseness. The same or similar parts may be marked with the same or similar reference numerals.

While various embodiments are described herein, it should be appreciated that the present invention encompasses many inventive concepts that may be embodied in a wide variety of contexts. The following detailed description of exemplary embodiments, read in conjunction with the accompanying drawings, is merely illustrative and is not to be taken as limiting the scope of the invention, as it would be impossible or impractical to include all of the possible embodiments and contexts of the invention in this disclosure. Upon reading this disclosure, many alternative embodiments of the present invention will be apparent to persons of ordinary skill in the art. The scope of the invention is defined by the appended claims and equivalents thereof.

Illustrative embodiments of the invention are described below. In the interest of clarity, not all features of an actual implementation are described in this specification. In the development of any such actual embodiment, numerous implementation-specific decisions may need to be made to achieve the design-specific goals, which may vary from one implementation to another. It will be appreciated that such a development effort, while possibly complex and time-consuming, would nevertheless be a routine undertaking for persons of ordinary skill in the art having the benefit of this disclosure.

Microfluidic lab-on-a-chip systems provide a versatile platform for rapid biosensing and environmental monitoring, because of various advantages associated with its integration, miniaturization, portability, and automation. Along with the advantage of high throughput, droplet microfluidic systems enable rapid mixing of fluids in the droplet microreactors with high reaction efficiency. In addition, the high surface-area-to-volume ratio from microfluidic droplets makes it promising in developing high-sensitivity interfacial biosensing between two different phases, thus overcoming the insolubility issues of many organic compounds like 17β-estradiol in various aqueous phase-based detection systems. Therefore, taking advantage of the high surface-to-volume property of microfluidic droplets, the present disclosure describes the development of a simple interfacial nano-biosensing strategy for high-sensitivity detection of low-solubility compounds like 17β-estradiol.

Many molecules such as estradiol and drugs have limited solubility in water, which affects their detection. Taking advantage of the property of high surface-area-to-volume ratio of microfluidic droplets, the present disclosure describes the development an innovative interfacial nanosensing strategy based on aptamer-functionalized graphene oxide nanosensors in microfluidic droplets for high-sensitivity one-step detection of low-solubility molecules. While estradiol is used as a model compound to demonstrate the proof-of-concept, it should be understood that the apparatuses, methods and compositions disclosed herein have broad applicability to any molecules that have low-solubility in any liquid such as water.

A number of hormones (e.g. estradiol), proteins (e.g. globulins and prolamines), drugs (approximately 40% of approved drugs are poorly water soluble, “Review of health benefits and business potentials.” OA Drug Design & Delivery 2013 Aug. 1; 10:4 vitamins (e.g. vitamin D, E and A), fats, polymers, and a lot of other organic compounds, are poorly water soluble. Therefore, apparatuses, methods, and compositions disclosed herein have wide applications in different fields related to these molecules, in particular and broadly with other “immiscible” fluids as described below.

In certain embodiments the methods and devices described herein may utilize immiscible fluids. In this context, the term “immiscible” when used with respect to two fluids indicates that the fluids when mixed in some proportion, do not form a solution. Classic immiscible materials are water and oil. Immiscible fluids, as used herein also include fluids that substantially do not form a solution when combined in some proportion. Commonly the materials are substantially immiscible when they do not form a solution if combined in equal proportions. In certain embodiments immiscible fluids include fluids that are not significantly soluble in one another, fluids that do not mix for a period of time due to physical properties such as density or viscosity, and fluids that do not mix for periods of time due to laminar flow. For example, solutions of water and 17β-estradiol are immiscible because 17β-estradiol is not significantly soluble in water.

In addition, such fluids are not restricted to liquids but may include liquids and gases. Thus, for example, where the droplets are to be formed comprising an aqueous solvent (such as water) any number of organic compounds such as carbon tetrachloride, chloroform, cyclohexane, 1,2-dichloroethane, dichloromethane, diethyl ether, dimethyl formamide, ethyl acetate, heptane, hexane, methyl-tert-butyl ether pentane, toluene, 2,2,4-trimethylpentane, and the like are contemplated. Various mutually insoluble solvent systems are well known to those skilled in the art (see e.g., Table 1). In another example, droplets of aqueous buffer containing physiologically normal amounts of solute may be produced in a dense aqueous buffer containing high concentrations of sucrose. In yet another example, droplets of an aqueous buffer containing physiologically normal amounts of solute may be produced in a second aqueous buffer containing physiologically normal amounts of solute where the two buffers are segregated by laminar flow. In still another example, droplets of a fluid may be produced in a gas such as nitrogen or air. In certain embodiments, either water-in-oil or oil-in-water droplets can be formed by two immiscible solvents.

Table 1 illustrates various solvents that are either miscible or immiscible in each other. The solvent on left column does not mix with solvents on right column unless otherwise stated.

| Solvents | Immiscibility |
|---|---|
| Acetone | can be mixed with any of the solvents listed in |
| | the column at left |
| Acetonitrile | cyclohexane, heptane, hexane, pentane, |
| | 2,2,4-trimethylpentane |
| carbon tetrachloride | can be mixed with any of the solvents listed in |
| | the column at left except water |
| chloroform | can be mixed with any of the solvents listed in |
| | the column at left except water |
| cyclohexane | acetonitrile, dimethyl formamide, dimethyl |
| | sulfoxide, methanol, water |
| 1,2-dichloroethane | can be mixed with any of the solvents listed in |
| | the column at left except water |
| dichloromethane | can be mixed with any of the solvents listed in |
| | the column at left except water |
| diethyl ether | dimethyl sulfoxide, water |
| dimethyl formamide | cyclohexane, heptane, hexane, pentane, |
| | 2,2,4-trimethylpentane, water |
| dimethyl solfoxide | cyclohexane, heptane, hexane, pentane, |
| | 2,2,4-trimethylpentane, diethyl ether |
| I,4-dioxane | can be mixed with any of the solvents listed in |
| | the column at left |
| ethanol | can be mixed with any of the solvents listed in |
| | the column at left |
| ethyl acetate | can be mixed with any of the solvents listed in |
| | the column at left except water |
| heptane | acetonitrile, dimethyl formamide, dimethyl |
| | sulfoxide, methanol, water |
| hexane | acetonitrile, dimethyl formamide, dimethyl |
| | sulfoxide, methanol, acetic acid, water |
| methanol | cyclohexane, heptane, hexane, pentane, |
| | 2,2,4-trimethylpentane |
| methyl-tert-butyl | can be mixed with any of the solvents listed in |
| ether | the column at left except water |
| pentane | acetonitrile, dimethyl formamide, dimethyl |
| | sulfoxide, methanol, water, acetic acid |
| I-propanol | can be mixed with any of the solvents listed in |
| | the column at left |
| 2-propanol | can be mixed with any of the solvents listed in |
| | the column at left |
| tetrahydrofuran | can be mixed with any of the solvents listed in |
| | the column at left |
| toluene | can be mixed with any of the solvents listed in |
| | the column at left except water |
| 2,2,4-trimethylpentane | acetonitrile, dimethyl formamide, dimethyl |
| | sulfoxide, methanol, water |
| water | carbon tetrachloride, chloroform, cyclohexane, |
| | I,2-dichloroethane, dichloromethane, diethyl |
| | ether, dimethyl formamide, ethyl acetate, heptane, |
| | hexane, methyl-tert-butyl ether, pentane, toluene, |
| | 2,2,4-trimethylpentane, estradiol |

U.S. Provisional Application 62/004,260, incorporated herein in its entirety, teaches a paper/polymer hybrid microfluidic device for simple one-step pathogen detection, using aptamer-functionalized graphene oxide (GO) nanosensors. Because of the quenching property of graphene oxide and simplicity offered by aptamers, this nanosensor system provides a simple method for one-step pathogen detection. However, it is not feasible to use this nanosensing system to detect estradiol due to the insolubility issue of estradiol. The combination of droplet microfluidics with aptamer-functionalized GO nanosensors enables a new interfacial nano-biosensing method for detection of low-insolubility organic compounds, with high simplicity and high sensitivity.

A biosensor as used herein can comprise an aptamer that specifically binds a target that is coupled to a reporter moiety and a quenching moiety, wherein the fluorescent moiety is quenched in the absence of a target molecule and when bound to a target molecule that quenching is suppressed or release. The biosensors of the composition may be specific for different target molecules, and may be associated with the same or different reporter molecules.

In certain aspects aptamers can be coupled to a variety of reporter moieties. Reporter moieties include fluorescent reporter moieties that can used to detect aptamer binding to a target. Fluorophores can be fluorescein isothiocyanate (FITC), allophycocyanin (APC), R-phycoerythrin (PE), peridinin chlorophyll protein (PerCP), Texas Red, Cy2, Cy3, Cy3.5, Cy5, Cy5.5, Cy7; or fluorescence resonance energy tandem fluorophores such as PerCPCy5.5, PE-Cy5, PE-Cy5.5, PE-Cy7, PE-Texas Red, and APC-Cy7. Other fluorophores include, Alexa Fluor® 350, Alexa Fluor® 488, Alexa 25 Fluor® 532, Alexa Fluor® 546, Alexa Fluor® 568, Alexa Fluor® 594, Alexa Fluor® 647; BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665; Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, and tetramethylrhodamine, all of which are also useful for fluorescently labeling aptamers.

Quenching refers to any process that decreases the fluorescence intensity of a given substance. A variety of processes can result in quenching, such as excited state reactions, energy transfer, complex-formation, and collisional quenching. Molecular oxygen, iodide ions, and acrylamide are common chemical quenchers. The chloride ion is a well-known quencher for quinine fluorescence. Typically quenching poses a problem for non-instant spectroscopic methods, such as laser-induced fluorescence, but can also be used in producing biosensors. In certain aspects the fluorescence of a labeled aptamer that is not bound to its target is quenched, wherein upon binding to its target the fluorescence is recovered and can be detected. The labeled aptamer is complexed with a quenching moiety forming a probe complex. Once the aptamer binds its target the fluorescence is recovered. Target binding results in increased fluorescence.

In certain aspects the fluorescence can be quenched by forming an aptamer/graphene oxide complex. Graphene oxide (GO) can act as a quencher of fluorescence and is easily dispersible in water.

In various embodiments, the droplets generated by the devices and methods described herein can contain or encapsulate a wide variety of materials. In some embodiments, the droplets may contain test samples, cells, organelles, proteins, nucleic acids, enzymes, PCR or other testing reagents, biochemicals, dyes, or particulates (for example polymeric microspheres, metallic microparticles, or pigments). In still other embodiments a droplet may encapsulate one or more previously generated droplets. In addition, the invention need not be limited to aqueous droplet systems. For example, such droplet generating methods and devices may be used in nanoparticle coating, where materials in organic solvents can be used to deposit layers on or encapsulate nanoparticles.

As noted above, in some embodiments, an opening in a fluid channel can be configured as a nozzle or other formats of microdroplet generators. The depth, inner diameter, and outer diameter of such a nozzle can be optimized to control droplet size, droplet uniformity, mixing at the fluid interface, or a combination of these.

In certain embodiments, the droplet generation and/or droplet merger components described herein may be provided on a substrate that differs from the material that comprises the fluid channels. For example, the fluid channels may be fabricated using an elastomeric material that is disposed upon a rigid surface. Suitable fluid channel materials include but are not limited to flexible polymers such as polydimethylsiloxane (PDMS), plastics, and similar materials. Fluid channels may also be comprised of nonflexible materials such as rigid plastics, glass, silicon, quartz, metals, and similar material. Suitable substrates include but are not limited to transparent substrates such as polymers, plastic, glass, quartz, or other dielectric materials. Other suitable substrate materials include but are not limited to nontransparent materials such as opaque or translucent plastics, silicon, metal, ceramic, and similar materials.

The parameters described above and in the examples (e.g., flow rate(s), laser intensity, laser frequency/wavelength, channel dimensions, port/nozzle dimensions, channel wall stiffness, location of cavitation bubble formation, and the like) can be varied to optimize droplet formation and/or droplet/particle/cell encapsulation for a particular desired application.

There are a number of formats, materials, and size scales that may be used in the construction of the droplet generating devices described herein and in microfluidic devices that may incorporate them. In some embodiments, the droplet generating devices and the connecting fluid channels are comprised of PDMS (or other polymers), and fabricated using soft lithography. PDMS is an attractive material for a variety of reasons, including but not limited to low cost, optical transparency, ease of molding, and elastomeric character. PDMS also has desirable chemical characteristics, including compatibility with both conventional siloxane chemistries and the requirements of cell culture (e.g., low toxicity, gas permeability). In an illustrative soft lithography method, a master mold is prepared to form the fluid channel system. This master mold may be produced by a micromachining process, a photolithographic process, or by any number of methods known to those with skill in the art. Such methods include, but are not limited to, wet etching, electron-beam vacuum deposition, photolithography, plasma enhanced chemical vapor deposition, molecular beam epitaxy, reactive ion etching, and/or chemically assisted ion beam milling (Choudhury, “The Handbook of Microlithography, Micromachining, and Micro fabrication,” Society Photo-Optical Instrument Engineer., Bard & Faulkner, Fundamentals of Microfabrication, 1997).

Once prepared the master mold is exposed to a pro-polymer, which is then cured to form a patterned replica in PDMS. The replica is removed from the master mold, trimmed, and fluid inlets are added where required. The polymer replica may be optionally treated with a plasma (e.g., an O₂ plasma) and bonded to a suitable substrate, such as glass. Treatment of PDMS with O₂ plasma generates a surface that seals tightly and irreversibly when brought into conformal contact with a suitable substrate, and has the advantage of generating fluid channel walls that are negatively charged when used in conjunction with aqueous solutions. These fixed charges support electrokinetic pumping that may be used to move fluid through the device. While the above described fabrication of a droplet generating device using PDMS, it should be recognized that numerous other materials can be substituted for or used in conjunction with this polymer. Examples include, but are not limited to, polyolefin elastomers, perfluoropolyethylene, polyurethane, polyimides, and cross-linked phenol/formaldehyde polymer resins.

In some embodiments, single layer devices are contemplated. In other embodiments, multilayer devices are contemplated. For example, a multilayer network of fluid channels may be designed using a commercial CAD program. This design may be converted into a series of transparencies that is subsequently used as a photolithographic mask to create a master mold. PDMS cast against this master mold yields a polymeric replica containing a multilayer network of fluid channels. This PDMS cast can be treated with a plasma and adhered to a substrate as described above.

As noted above, the apparatuses, methods, and compositions disclosed herein are particularly suitable for use in microfluidic devices. In some embodiments therefore the fluid channels are microchannels. Such microchannels have characteristic dimensions (height or depth, width, or length) ranging from about 100 nanometers to 1 micron up to about 1000 microns. In various embodiments the characteristic dimension ranges from about 1, 5, 10, 15, 20, 25, 35, 50 or 100 microns up to about 150, 200, 250, 300, or 400 microns. In some embodiments, the characteristic dimension ranges from about 20, 40, or about 50 microns up to about 100, 125, 150, 175, or 200 microns. In various embodiments, the wall thickness between adjacent fluid channels ranges from about 0.1 micron to about 50 microns, or about 1 micron to about 50 microns, more typically from about 5 microns to about 40 microns. In certain embodiments, the wall thickness between adjacent fluid channels ranges from about 5 microns to about 10, 15, 20, or 25 microns.

In various embodiments the depth of a fluid channel ranges from 5, 10, 15, 20 microns to about 1 mm, 800 microns, 600 microns, 500 microns, 400 microns, 300 microns, 200 microns, 150 microns, 100 microns, 80 microns, 70 microns, 60 microns, 50 microns, 40 microns, or about 30 microns. In certain embodiments, the depth of a fluid channel ranges from about 10 microns to about 60 microns, more preferably from about 20 microns to about 40 or 50 microns. In some embodiments, the fluid channels can be open; in other embodiments, the fluid channels may be covered.

Microdevices described herein can comprise one or more microwells configured for sample detection. In certain aspects one or more microwells are in fluid communication with one or more microchannels and/or reservoirs. In certain aspects a microwell can comprise a paper based biosensor for the direct or indirect detection of one or more compounds that have low solubility in water. In certain aspect one well can be a reaction well and a second well a detection well. Each of the wells can be reversibly sealed to form a chamber. In another aspect the microchannel can be modified to form a reaction or detection zone that acts on a sample as it flows through the zone.

As noted above, in some embodiments a nozzle is present. In certain embodiments, where a nozzle is present, the nozzle diameter can range from about 0.1 micron, or about 1 micron up to about 300 microns, 200 microns, or about 100 microns. In certain embodiments, the nozzle diameter can range from about 5, 10, 15, or 20 microns up to about 25, 30, 35, 40, 45, 50, 55, 60, 65, 70, 75, or about 80 microns. In some embodiments, the nozzle diameter ranges from about 1, 5, 10, 15, or 20 microns to about 25, 35, or 40 microns.

In some embodiments, the methods and devices described herein can generate droplets at a rate ranging from zero droplets/sec, about 2 droplets/sec, about 5 droplets/sec, about 10 droplets/sec, about 20 droplets/sec, about 50 droplets/sec, about 100 droplets/sec, about 500 droplets/sec, or about 1000 droplets/sec, up to about 1,500 droplets/sec, about 2,000 droplets/sec, about 4,000 droplets/sec, about 6,000 droplets/sec, about 8,000 droplets/sec, about 10,000 droplets/sec, about 20,000 droplets/sec, about 50,000 droplets/sec, and about 100,000 droplets/sec.

In various embodiments, the apparatuses, methods and compositions described herein can generate droplets having a substantially continuous volume. Droplet volume can be controlled to provide volumes ranging from about 0.1 fL, about 1 fL, about 10 fL, and about 100 fL to about 1 microliter, about 500 nL, about 100 nL, about 1 nL, about 500 pL or about 200 pL. In certain embodiments volume control of the droplet ranges from about 1 pL to about 150 pL, about 200 pL, about 250 pL, or about 300 pL.

As indicated above, the microchannel droplet formation/merger injection devices described herein can provide a system integrated with other processing modules on a microfluidic “chip” or in flow through fabrication systems for microparticle coating, microparticle drug carrier formulation, and the like. These uses, however, are merely illustrative and not limiting.

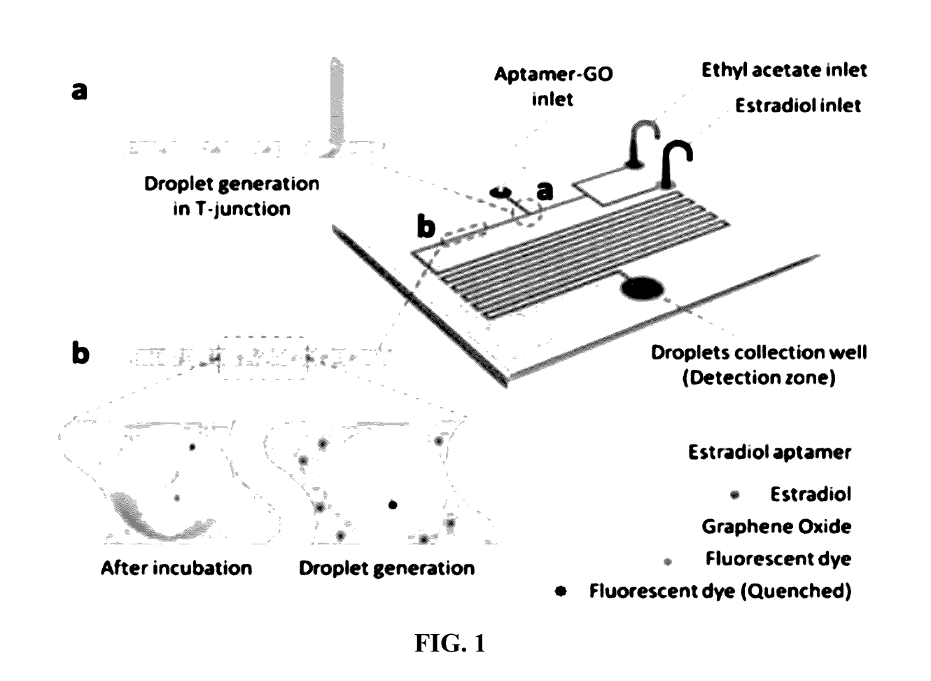

In one aspect of the disclosure, microfluidic chip design and the detection principle of interfacial nano-biosensing strategy are depicted in FIG. 1. FIG. 1 illustrates a schematic of the droplet microfluidic system for one-step estradiol detection using aptamer-functionalized GO nanosensors. The high surface-to-volume ratio from droplet microfluidics enables high-sensitivity interfacial nano-biosensing. Portions (a) and (b) of the chip channel are magnified at inset (a) and (b) to show the detection principle, respectively.

The chip has two layers. The top layer is a polydimethylsiloxane (PDMS) layer consisting of three inlets for the delivery of estradiol, ethyl acetate, and the aptamer-GO complex, microchannels, and an outlet for droplet collection. The outlet region is also used as the detection zone. A glass layer at the bottom is used for structural support. The T-junction method for microfluidic droplet generation is used, as depicted at inset (a) of FIG. 1.

Estradiol is dissolved in ethyl acetate as the oil phase, whereas an aqueous solution of aptamer-functionalized graphene-oxide nanosensors are used as the aqueous phase. However, it should be understood that any oil-phase solvent besides ethyl acetate known in the art may be used and is contemplated by the disclosure herein. Because of the extraordinary distance-dependent fluorescence quenching property of GO, fluorescence of the Cy3-labeled aptamer will be pre-quenched in the aptamer-GO aqueous phase (fluorescence ‘off’; as depicted at inset (b) of FIG. 1). By changing different flow rates of ethyl acetate and estradiol, different concentrations of estradiol solutions can readily be detected by this droplet microfluidic system. When the water phase and the oil phase introduced at different flow rates meet at the T-junction, water-in-oil emulsion droplets will be generated due to the shear stress from the continuous oil flow which is set at a higher flow rate (2.4 μL/min) than the water phase (0.6 μL/min).

It should be understood that the probe or aptamer can be complexed with other materials including, but not limited to, graphene, graphene oxide (GO), and/or other carbon nanoparticles known in the art, to form a biosensor. In certain aspects the fluorescence of aptamers and probes can be quenched by graphene oxide, graphene, and/or carbon nanoparticles. In certain aspects the aptamer/grapheme oxide complex is adsorbed to a substrate or layer. Graphene oxide (GO) is a compound of carbon, oxygen, and hydrogen in variable ratios, obtained by treating graphite with strong oxidizers. Graphene oxide (GO) is an intermediate on the route to chemically derived graphene, and it is easily synthesized. Its chemical structure is heterogeneous and consists of both large areas of conjugated sp2-systems and various electronically isolated oxygen containing functionalities. GO can act as a quencher of fluorescence and is easily dispersible in water. In some instances the binding of the target results in desorption of the aptamer, which in turn results in an increase in fluorescence.

In another aspect of the present disclosure, droplet generation includes the following materials and methods.

Chemicals and Materials:

17β-estradiol and ethyl acetate were purchased from Sigma (St. Louis, Mo.). Graphene oxide was purchased from Graphene Laboratories (Calverton, N.Y.). While the quencher described herein is graphene oxide, it is contemplated by the present disclosure that other quenchers known in the art, e.g., graphene may be used. In certain aspects a quenching moiety is manganese or, graphene, graphene oxide and carbon nanoparticles.

Polydimethylsiloxane (PDMS, Sylgard 184) was obtained from Dow Corning (Midland, Mich.). All other chemicals were purchased from Sigma (St. Louis, Mo.) and used without further purification, unless stated otherwise. Unless otherwise noted, all solutions were prepared with ultrapure Milli-Q water (18.2 MΩ·cm) from a Millipore Milli-Q system (Bedford, Mass.).

The sequence of the cy3 fluorescence labeled estradiol aptamer (Integrated DNA technologies, Coralville, Iowa) was listed as the following (76 mer, 5′-3′): Cy3-GCTTCCAGCTTATTGAATTACACGCAGAGGGTAGCGGCTCTGCGCATTCAATTGCTG CGCGCTGAAGCGCGGAAGC (SEQ ID NO:1). This estradiol aptamer (Kim et al., Biosensors and Bioelectronics, 2007, 22:2525) is preferred for this embodiment but any other conventional aptamer that binds to a compound (or molecule) of interest may be used and is contemplated within the scope of this disclosure. Aptamers may be in the form of an oligonucleotide or peptide molecules that bind to a specific target molecule. Aptamers are usually created by selecting them from a large random sequence pool, but natural aptamers also exist in the form of riboswitches. Aptamers may be combined with ribozymes to self-cleave in the presence of their target molecule. Aptamers may be classified as DNA or RNA or XNA aptamers which usually consist of (usually short) strands of oligonucleotides or peptide aptamers which consist of a short variable peptide domain, attached at both ends to a protein scaffold. For example, the aptamers could be any fluorophore-labelled DNA oligonucleotides, e.g., DNA capture probes.

Microfluidic System Fabrication:

PDMS microfluidic devices were molded through a Silicon master. Briefly, a thin layer of chrome (50 nm, RF sputtered) was used as a mask on a 4″ wafer. Then the design was lithographically transferred using 1813 PR (photoresist), after developing the PR and 100-second etching Cr with Chrome etchants. Using a Plasmalab-100 System from Oxford Instruments, a DRIE BOSCH process was used to etch Silicon by 45 microns. The DRIE process used 150 steps to etch through Silicon, and each step etched 30 nm of silicon each 12 seconds.

PDMS films were prepared following standard soft lithography procedures. Firstly, the liquid PDMS base and the curing agent were mixed at a weight ratio of 10:1. Then the PDMS precursor mixture was poured onto the silicon wafer, degassed in a vacuum desiccator for approximately 30 minutes, and incubated at 95° C. for 2 hours. The channel width was about 60 μm. Inlet reservoirs in the top PDMS layer and outlet reservoirs were excised using biopsy punches. After 30 seconds exposure in an oxidizing air Plasma Cleaner (Ithaca, N.Y.), PDMS films and the glass slide were face-to-face sandwiched to bond irreversibly. Thus, the biochip became ready for use.

Aptamer-GO Preparation:

GO was diluted in Milli-Q water and was then mixed with the fluorescent aptamer solution at a final concentration of 0.04 mg/mL. The aptamer-functionalized GO was incubated for 15 minutes to quench the fluorescence of the aptamer, and the optimal quenching time was investigated by introducing aptamer-functionalized GO into detection wells on the chip. As described herein, a resulting composition generates an integrated one-step aptamer/probe-functionalized graphene oxide (GO) biosensor(s) on a chip, using a sensitive “turn on” strategy based on the fluorescence quenching and recovering propriety of GO when adsorbing and desorbing fluorescent labeled aptamers or probes.

Droplet Generation:

Droplets were generated by using a T-junction method. Aptamer-GO was used as the water phase and estradiol in the ethyl acetate solvent was used as the oil phase with flow speed of 0.6 μL/min and 2.4 μL/min, respectively. The droplet generation process was demonstrated in by using the Cy3-labelled aptamer. After 30 min incubation at room temperature, droplets were detected at the outlet region by a Nikon Ti-E microscope (Melville, N.Y.) with appropriate Cy3 optical filters. Cy3 is a cyanine dye that fluoresces greenish yellow (˜550 nm excitation, ˜570 nm emission). However, as known in the art, any fluorescent dye with additional modifications and any optical filter for dye detection may be used and is contemplated within the scope of this disclosure.

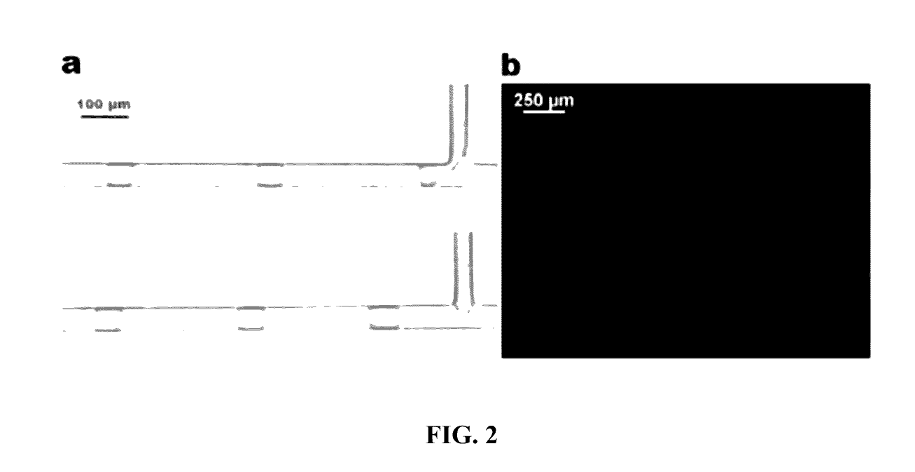

FIGS. 2A-2B depict a series of images during and after the droplet generation process. Specifically, FIG. 2A depicts captured images during the droplet generation process by using the T-junction method. Food dye was added to distinguish droplets from the continuous flow. FIG. 2B is the fluorescence image after droplet generation with Cy3-labeled aptamer in droplets.

After water-in-oil droplet generation as described above, aptamer-functionalized GO nanosensors in aqueous droplets will start to react with the target of estradiol from the oil phase at the droplet interface between these two immiscible phases. The large surface from millions of droplets significantly enhances the interaction possibilities between aptamer-GO nanosensors with the target. In the presence of the target, the aptamer will bind specifically to the corresponding target estradiol. The competitive binding of the aptamer and target estradiol lowers affinity of the adsorption with GO and spontaneously liberates the aptamer from the GO surface, thus resulting in the fluoresce recovery (fluorescence ‘turn-on’; see inset (b) of FIG. 1). After 30-min incubation, recovered fluorescence is detected by a fluorescence microscope at the outlet region. No fluorescence restoration is observed in the absence of the target. Hence, the aptamer-functionalized GO nanosensors in droplets enables a simple one-step “turn on” mechanism for high-sensitivity estradiol detection. As such, the present disclosure describes the novel use of the large effective area of microfluidic droplets to develop high-sensitivity nano-biosensing system based on enhanced interfacial reactions.

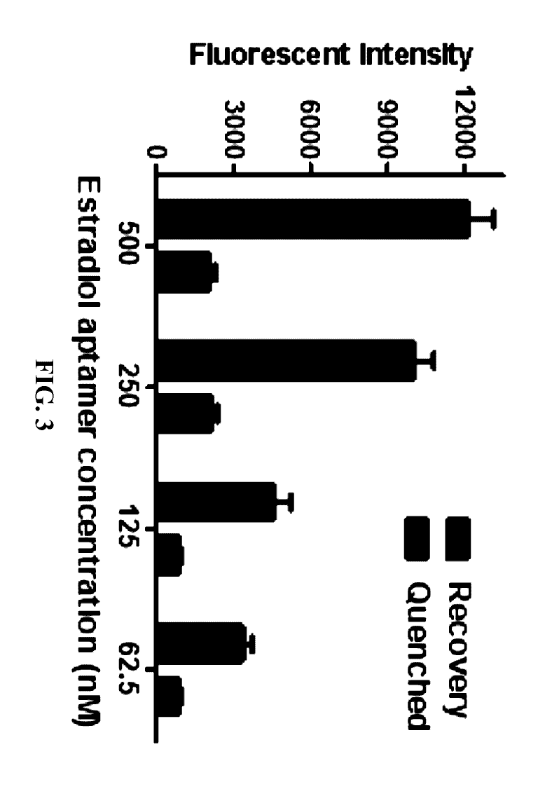

In another aspect of the disclosure, optimization of the aptamer concentration is critical for high-sensitivity detection of estradiol. Therefore, four different concentrations of the aptamer ranging from 62.5 to 500.0 nM were tested to optimize the aptamer concentration for the droplet microfluidic system by using 1000.0 pM estradiol. The corresponding fluorescent intensities at different aptamer concentrations after quenching and recovery are shown in FIG. 3. Specifically, FIG. 3 illustrates the results of estradiol aptamer concentration optimization wherein the estradiol concentration is 1000.0 pM. Two important factors, both the recovered fluorescent intensity and the net fluorescence increase (i.e., the difference between the recovered and quenched fluorescent intensity) need to be considered since they can directly affect the detection sensitivity.

From FIG. 3 it can be discerned that the fluorescence of the aptamer were significantly quenched by GO for all aptamer concentrations, without significant differences between different concentrations. However, the restored fluorescence varied greatly at different aptamer concentrations, ranging from 3700 to 12300 a.u. corresponding to the aptamer concentrations from 62.5 nM to 500.0 nM. 500.0 nM of the aptamer exhibited the maximal net fluorescence recovery (approximately 7 folds increase). At lower aptamer concentrations, the recovered fluorescent intensities were much lower. Given the highest recovered fluorescence intensity and maximal difference between the recovered and quenched fluorescent intensity, 500.0 nM was selected as the aptamer concentration for the subsequent experiments.

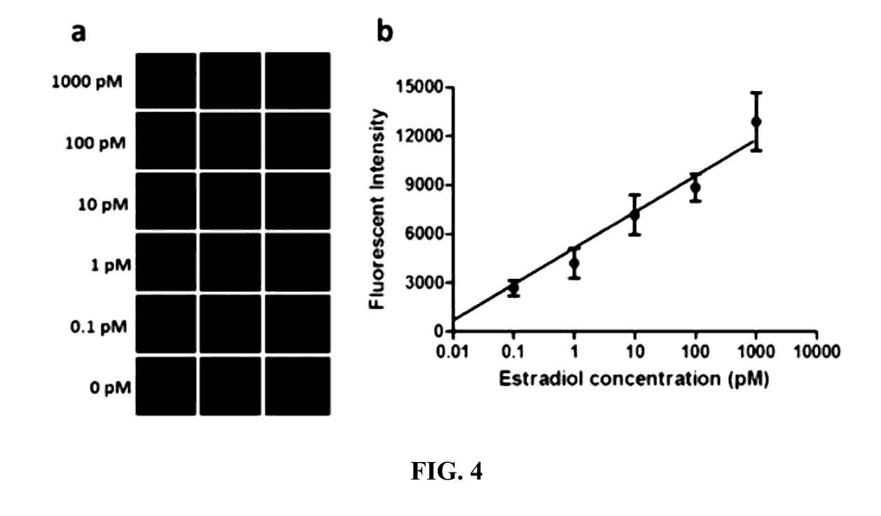

In another aspect of the disclosure, under the optimized aptamer conditions, the detection of estradiol using different concentration of standards were tested with their corresponding recovered fluorescence intensities recorded. FIGS. 4A-4B depict fluorescence images (FIG. 4A) and calibration curve (FIG. 4B) of the detection of different concentrations of estradiol by using droplet microfluidic nanosensing system. The estradiol aptamer concentration was 500.0 nM.

Specifically, FIGS. 4A-4B illustrate the recovered fluorescent images and the calibration curve plotted by using recovered fluorescence intensities versus various concentrations of estradiol ranging from 0.1 pM to 1000 pM. Compared to the negative control (0 pM of estradiol), even 0.1 pM of estradiol showed well distinguishable fluorescence signal. With the increase of estradiol concentration, stronger recovered fluorescence intensity was observed. As can be discerned in FIG. 4B, a linear calibration curve was established between the recovered fluorescence and the estradiol concentration, with the square of the correlation coefficient (i.e., R²) of 0.997. The Limit Of Detection (LOD) of estradiol was calculated to be as low as 0.07 pM on the basis of the 3-fold standard deviations of the negative control signal, whereas the LODs of most aptamer-based biosensors were in the range of nM (e.g., 2.1 nM) or above the pM range (e.g., 100 pM and 2.0 pM). It is noteworthy to mention that the reported LODs from two previous conventional methods using the same aptamer and the same optical detection method of fluorescence are 0.22 nM and 2.1 nM. Hence, the LOD is 2000 and 20000 folds lower than these two conventional methods, respectively, indicating the high sensitivity of the interfacial nano-biosensing methods using microfluidic droplets of the present disclosure. This LOD from using the droplet microfluidics disclosed in the present invention is the lowest reported for estradiol detection. This is the first time to use the large effective area of microfluidic droplets to develop high-sensitivity nano-biosensing system based on enhanced interfacial reactions.

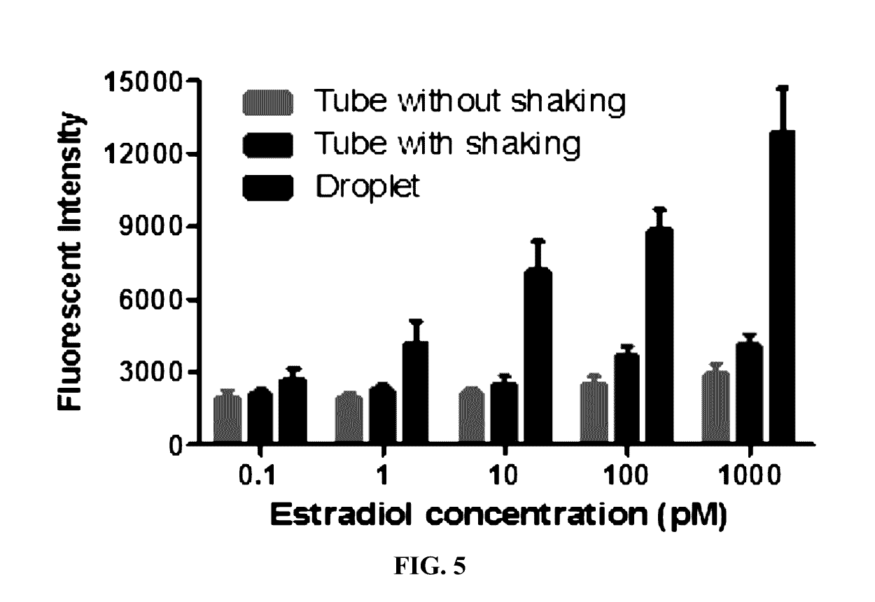

In yet another aspect of the disclosure, the sensitivity of the droplet microfluidic nanosensing system of the present disclosure is compared with conventional off-chip methods by testing various concentrations of estradiol. During the off-chip detection, the mixed aptamer-GO and estradiol solutions were incubated at room temperature for 30 min in different microtubes with continuous shaking and without shaking, respectively. The fluorescent intensities generated by our droplet microfluidic method and these two conventional off-chip methods were recorded. Their comparison is shown in FIG. 5. FIG. 5 is a comparison of estradiol detection results between the on-chip system of the present disclosure and two conventional off-chip detection methods wherein estradiol aptamer concentration is 500 nM. For the off-chip detection method without shaking, there were no obvious enhanced fluorescent intensities after incubation at all estradiol concentrations; for the off-chip detection method with shaking, slightly enhanced fluorescent intensities were observed at only higher estradiol concentrations (>100 pM), indicating the low performance of the off-chip detection methods.

It is estimated that the LODs of estradiol from these two conventional methods with and without shaking are about 150.0 and 20.0 pM, respectively. The LOD of 150.0 pM is consistent with previously published value of 0.22 nM. Although shaking can lower the LOD down to 20.0 pM, its LOD is still about 200 folds higher than that of our interfacial nano-biosensing system. Compared with the off-chip detection methods, the on-chip detection method of the present disclosure showed high performance with much higher fluorescent intensities, because the droplet system greatly increased the reaction kinetics and efficiency between the two phase interfaces due to enhanced effective contact areas in droplets.

Taking advantage of the large effective surface area from microfluidic droplets, the present disclosure teaches the development of an interfacial nano-biosensing strategy based on aptamer-functionalized GO nanosensors in droplets for high-sensitivity one-step 17β-estradiol detection. The LOD was calculated to be as low as 0.07 pM. The detection sensitivity for estradiol has been improved by about 3 orders of magnitude over other conventional systems.

While 17β-estradiol was used as the initial test compound, other compounds found in any environment and in biological fluids may be similarly detected. This study should have great potential for high-sensitivity food safety and environmental monitoring. This interfacial nano-biosensing system can also be used to solve the detection problems of many low-solubility compounds in numerous aqueous solutions-based detection systems.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment,” “in another embodiment,” or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Similarly, although example processes have been described with regard to particular operations performed in a particular sequence, numerous modifications could be applied to those processes to derive numerous alternative embodiments of the present invention. For example, alternative embodiments may include processes that use fewer than all of the disclosed operations, processes that use additional operations, and processes in which the individual operations disclosed herein are combined, subdivided, rearranged, or otherwise altered.

This disclosure may include descriptions of various benefits and advantages that may be provided by various embodiments. One, some, all, or different benefits or advantages may be provided by different embodiments. In view of the wide variety of useful permutations that may be readily derived from the example embodiments described herein, this detailed description is intended to be illustrative only, and should not be taken as limiting the scope of the invention. What is claimed as the invention, therefore, are all implementations that come within the scope of the following claims, and all equivalents to such implementations.

What is claimed is:

1. A method for detecting water immiscible compounds, comprising:
fabricating at least one microfluidic chip;
generating microfluidic droplets by the addition of a sample, at least one oil-phase solvent, and an aptamer nanosensor complex;
allowing competitive binding between the aptamer nanosensor complex and the sample; and,
measuring the level of fluorescence released following the competitive binding.

2. The method of claim 1, wherein the microfluidic chip comprises two layers.

3. The method of claim 2, wherein the first layer is a polymer.

4. The method of claim 3, wherein the polymer is polydimethylsiloxane (PDMS).

5. The method of claim 4, wherein the first layer further comprises at least one inlet for the delivery of the sample, at least one inlet for delivery of the at least one oil-phase solvent and at least one inlet for delivery of the aptamer nanosensor complex, a plurality of microchannels, and an outlet for droplet collection.

6. The method of claim 1, wherein the aptamer nanosensor complex is an aptamer-GO complex.

7. The method of claim 1, wherein the aptamer is complexed with graphene oxide, graphene or carbon nanoparticles to form a sensor.

8. The method of claim 2, wherein the second layer is glass.

9. The method of claim 1, wherein the sample comprises 17β-estradiol.

10. The method of claim 1, wherein the at least one oil-phase solvent is ethyl acetate.

11. An apparatus for detecting water immiscible compounds, comprising:
means for fabricating at least one microfluidic chip;
means for generating microfluidic droplets by the addition of a sample, ethyl acetate and an aptamer nanosensor complex;
means for allowing competitive binding between the aptamer nanosensor complex and the sample;
means for measuring the level of fluorescence released following the competitive binding.

12. A composition for detecting water immiscible compounds comprising an aptamer-GO complex bound to a sample.

13. The composition of claim 12, wherein the sample comprises 17β-estradiol.


SEQUENCE LISTING (1 sequence)

SEQ ID NO: 1 - DNA, 76 bp, Artificial Sequence
  Synthetic Primer
gcttccagcttattgaattacacgcagagggtagcggctctgcgcattcaattgctgcgcgctgaagcgcggaagc